(19) United States
(12) Patent Application Publication
(10) Pub. No.: US 2020/0179426 A1
(43) Pub. Date: Jun. 11, 2020

(54) METHODS OF USE FOR OENOTHEIN A AND B FROM EPILOBIUM SPECIES

(71) Applicant: EPILOBIUM, INC., SAN DIEGO, CA (US)

(72) Inventor: Robert Jacquet, San Diego, CA (US)

(21) Appl. No.: 16/788,032

(22) Filed: Feb. 11, 2020

Related U.S. Application Data
(63) Continuation-in-part of application No. 15/917,371, filed on Mar. 9, 2018.
(60) Provisional application No. 62/469,355, filed on Mar. 9, 2017.
(60) Provisional application No. 62/469,435, filed on Mar. 9, 2017.

(51) Int. Cl. A61K 31/7048; A61K 9/00; A61K 9/48; A61K 31/05; A61K 31/352; A61K 36/76; A61K 9/12; A61P 31/00; A61P 13/10; A61P 29/00

(52) U.S. Cl. CPC A61K 31/7048; A61K 9/0053; A61K 9/4808; A61K 31/05; A61K 31/352; A61K 36/76; A61K 9/4841; A61K 9/0073; A61K 9/0014; A61P 31/00; A61P 13/10; A61P 29/00; A61K 9/12

(57) ABSTRACT

The invention provides medicaments comprising an oenothein, including oral formulations to treat inflammation or to treat hormone balance in perimenopausal, menopausal and postmenopausal women. The oenothein for use in such formulations, such as oenothein A and oenothein B, maybe purified from natural sources, such as Epilobium, and used in combination with a cannabinoid, such as cannabidiol (CBD), cannabigerol (CBG) or tetrahydrocannabinol (THC).

### CROSS-REFERENCE TO RELATED APPLICATIONS

This application is a Continuation-In-Part of U.S. application Ser. No. 15/917,371, filed on Mar. 9, 2018, which claims priority under 35 USC § 119(e) to U.S. Application Ser. No. 62/469,355 filed on Mar. 9, 2017 and U.S. Application Ser. No. 62/469,435 filed on Mar. 9, 2017. The disclosure of the prior application is considered part of and is incorporated by reference in the disclosure of this application in its entirety.

### BACKGROUND OF THE INVENTION

### Field of the Invention

The present invention relates generally to compositions and uses for oenothein, specifically the invention relates to the use of oenothein A and B in combination with cannabinoids or cannabis for the treatment of various disease and disorders.

### Background Information

In the United States, the National Association for Continence (NAFC) reported in a nationwide survey conducted in 2002 that nearly one in four women age 20 to 70 years old had incontinence. Incontinence can be decreased by inhibiting the aromatase enzyme; as it may make more testosterone available to strengthen the pelvic muscles.

Incontinence occurs because of problems with muscles and nerves that help to hold or release urine. The body stores urine, water and wastes removed by the kidneys in the bladder, connected to the urethra. In a study of postmenopausal women, it was shown that decreased androgen (testosterone) levels weaken the pelvic floor and sphincter muscles, while an estrogen deficit induces atrophy of the urethra. With ages standardized, the prevalence of urinary incontinence in combined surveys was 51.1% in women and 13.9% in men. Prevalence in women increased from 49.5% in 2001 to 2002, to 53.4% in 2007 to 2008 (Ptrend=0.01) and in men from 11.5% to 15.1%, respectively.

Methicillin-resistant Staphylococcus aureus (MRSA) refers to a group of Gram-positive bacteria that are genetically distinct from other strains of Staphylococcus aureus. Worldwide, an estimated 2 billion people carry some form of S. aureus; of these, up to 53 million (2.7% of carriers) are thought to carry MRSA. MRSA is responsible for several difficult-to-treat infections in humans, as it is characterized by multiple drug resistance to beta-lactam antibiotics, a broad-spectrum group that include some penams (penicillin derivatives such as methicillin and oxacillin) and cephems such as the cephalosporins.

Epilobium is a plant used in many countries around the world, for the most part it is used in the treatment of prostate, bladder disorders (all classes) and for the tonification of the urinary tract. Research has shown that Epilobium contain therapeutic levels of oenothein A and B capable of inhibiting the conversion of testosterone to dihydrotestosterone (DHT) by its activity on 5-alpha-reductase and aromatase (the group of enzymes that catalyze the conversion of testosterone to estradiol).

A purified aqueous extract was obtained from New World Epilobium angustifolium (Canadian Willow herb) and subjected to a variety of analytical techniques, which unambiguously identify the purified compound as oenothein B. This analysis included mass spectroscopy (MS) and nuclear magnetic resonance spectrometry (NMR).

While oenothein B was found by these methods to be present in solvent-free aqueous extracts of Canadian Willow herb at levels up to 9.6%, myricetin glucuronide—the substance identified as the active ingredient of the anti-inflammatory action of Old World Epilobium angustifolium) was not found in extracts of Canadian Willow herb. (see U.S. Pat. No. 6,528,490).

Oenothein is a tannin found in a number of trees, plants and herbs. The natural product oenothein A and B, a dimeric macrocyclic ellagitannin, has a wide range of biological activities, such as antioxidant, anti-microbial, anti-inflammatory, antiviral, antifungal, and antitumor activity.

Oenothein has been described as a potential active agent for:

treating cardiovascular disease enhanced with acetylsalicylic acid as a polypill, for men and women;

treating joint pain and minor arthritis enhanced with Sodium Naproxen as a polypill for both men and women;

treating benign prostatic hyperplasia (BPH) for men, more specifically to the administration of oenothein ellagitannins A and or B, in subjects having or at risk of having such disorders; and

treating smooth muscle i.e., detrusor, as it pertains to hormone balance effecting bladder the vascular system and urinary tract.

Oenothein A and B have been included in the production of formulations for the treatment of diseases such as Androgenic Alopecia (AGA), hair loss for women (female pattern baldness). Oenothein A and B were formulated in a combination with a ingestible; or in formulation containing between 0.01%-100% oenothein B for topical application onto affected areas of the human scalp or as a prophylactic. It can be dissolved in suspension with liquid, gel, foam or other topical applications containing melatonin at 0.01 mg/per ml-0.05 mg/per ml, for topical application for women's hair loss

Oenothein A and B can be extracted using alcohol derived from sugar cane, molasses, vegetable, fruit, grain or other organic plant sources; using glycerol extracted from plant; or using water, alcohol and water, distilled or reverse osmosis (RO) and/or water infused with suspended antibacterial, antimicrobial elements including silver sub-nano/nano clusters. Silver nanoparticles constitute a very promising approach for the development of new antimicrobial systems. Nanoparticulate objects can bring significant improvements in the antibacterial activity of this element, through specific effect such as an adsorption at bacterial surfaces. Oenothein A and B can be extracted from Epilobium with alcohol or alcohol and water, where the alcohol is removed or separated mechanically, chemically, gravity, crystallization or with adequate temperature to distill the alcohol into a separate recovery vessel.

Ellagitannins are a diverse family of naturally-occurring compounds consisting of a central core of glucose esterified with hexahydroxydiphenic acid. Ellagitannins are known as active constituents of a variety of medicinal plants. For example, U.S. Pat. No. 5,843,911 discloses the use of ellagitannins having galloyl and hexahydroxydiphenoyl substituent's as hyaluronidase enzyme inhibitors for topical application, to enhance water retention in skin by preventing the hydrolysis of hyaluronic acid. The ellagitannins disclosed in U.S. Pat. No. 5,843,911 is identified as “GOD-type” ellagitannins, extractable from a variety of plants.

U.S. Pat. No. 5,525,594 discloses the use of the ellagitannins oenothein-B (which is not a “GOD-type” ellagitannin as defined in U.S. Pat. No. 5,843,911) for the treatment of hyper androgenic disorders by oral, rectal or parenteral administration. In this treatment, the mechanism of action of oenothein B is thought to be the inhibition of 5-reductase, an enzyme responsible for the conversion of testosterone to dihydrotestosterone. Both oenothein-A and oenothein-B from Epilobium species have been shown to have similar pharmacologic activity, evidenced by 5-reductase and aromatase inhibition. It is disclosed in U.S. Pat. No. 5,525,594 that oenothein-B may be extracted from various Onagraceae plants (evening-primrose family), including Epilobium parviflorum. Ducrey et al., 1996, supra, disclose the extraction of oenothein A and B from E. capense, and the quantization of oenothein-B in a variety of old world Epilobium species including Old World E. angustifolium L (Rosebay Willow herb). The apparent antiviral and antitumor activities of oenothein-B are also discussed by Ducrey et al., 1996, supra, and in U.S. Pat. No. 5,525,594.

Aqueous extracts of Old World Epilobium angustifolium have been suggested for use as oral anti-inflammatories, and the active ingredient in such extracts has been identified as the flavonoids compound myricetin glucuronide (3,3′4′,5,5′,7-hexahydroxyflavone-3-O-glucuronide).

It has previously been disclosed, by the present inventor, that crude aqueous extracts of New World Epilobium parviflorum (Small Willow herb) have anti-inflammatory properties, aromatase inhibition, DHT blocking, inhibit cyclooxygenase-1 and -2 (COX-1 and COX-2) catalyzed prostaglandin biosynthesis. Studies on antibacterial, anti-inflammatory and antioxidant activity of herbal remedies used in the treatment of benign prostatic hyperplasia and prostatitis.

Cannabis is a genus of flowering plants in the family Cannabaceae, also as hemp, more often used to refer only to varieties of Cannabis cultivated for non-drug use. Cannabis has long been used for hemp fiber, hemp seeds and their oils, hemp leaves for use as vegetables and as juice, medicinal purposes, and as a recreational drug. Cannabis plants produce a group of chemicals called cannabinoids, which produce mental and physical effects when consumed, and which have increased their study and use for medicinal purposes.

Cannabinoids, terpenoids, and other compounds are secreted by glandular trichomes that occur most abundantly on the floral calyxes and bracts of female plants. A cannabinoid is one of a class of diverse chemical compounds that acts on cannabinoid receptors, which are part of the endocannabinoid system found in cells that alter neurotransmitter release in the brain. Ligands for these receptor proteins include the endocannabinoids produced naturally in the body by animals; phytocannabinoids, found in cannabis; and synthetic cannabinoids, manufactured artificially. The most notable cannabinoid is the phytocannabinoid tetrahydrocannabinol (THC), the primary psychoactive compound in cannabis. Cannabidiol (CBD) is another major constituent of the plant. Cannabigerol (CBG), is one of the at least 113 different cannabinoids isolated from cannabis, exhibiting varied effects.

The present invention relates to the administration of oenothein ellagitannins specifically oenothein A and B in combination with a cannabinoid, such as cannabidiol (CBD), cannabigerol (CBG) or tetrahydrocannabinol (THC), for the treatment of a variety of diseases and conditions, including symptoms of overactive bladder, inflammation, hormonal imbalance, and MRSA infection.

### SUMMARY OF THE INVENTION

The invention is based on the seminal discovery that the administration of oenothein A or B in combination with a cannabinoid, such as cannabidiol (CBD), cannabigerol (CBG) or tetrahydrocannabinol (THC) is useful for treating a variety of disorders.

In one embodiment, the present invention provides a method of treating one or more symptoms of overactive bladder in a subject including administering to the subject a therapeutically effective amount of a composition including oenothein A or and B, and a cannabinoid, wherein the one or more symptoms of overactive bladder are selected from the group consisting of frequency of or urgency, nocturia, increase in urinary micturition frequency, urinary incontinence, hormone balance for perimenopausal, menopausal, postmenopausal women and inflammation, and wherein the cannabinoid is cannabidiol (CBD), tetrahydrocannabinol (THC), cannabigerol (CBG), or a combination thereof.

In another embodiment, the present invention provides a composition formulated for oral or topical delivery consisting essentially of oenothein A, oenothein B, and a cannabinoid, wherein the cannabinoid is cannabidiol (CBD), tetrahydrocannabinol (THC), cannabigerol (CBG), or a combination thereof.

In an additional embodiment, the invention provides a method of reducing inflammation, treating urinary incontinence, treating bacterial urinary incontinence, treating hormonal imbalance in perimenopausal, menopausal and postmenopausal women, treating female hair loss due to perimenopause, menopause, and/or postmenopause, treating the conversion of testosterone to estrogen (aromatase) in perimenopausal, menopausal, postmenopausal women, inhibiting cyclooxygenase-1 and -2 (COX-1 and COX-2) catalyzed prostaglandin biosynthesis, including administering to a subject in need thereof a therapeutically effective amount of a composition consisting essentially of oenothein A, oenothein B, and a cannabinoid, wherein the cannabinoid is cannabidiol (CBD), tetrahydrocannabinol (THC), cannabigerol (CBG), or a combination thereof.

In yet another embodiment, the invention provides a method of treating methicillin-resistant Staphylococcus aureus (MRSA) infection in a subject including administering to the subject a therapeutically effective amount of a composition consisting essentially of oenothein A, oenothein B, and cannabigerol (CBG).

### BRIEF DESCRIPTION OF THE DRAWINGS

FIG. 1 is a drawing of the known structure of oenothein-A.

FIG. 2 is a drawing of the known structure of oenothein-B.

FIG. 3 is a listing of active ingredients particular of Epilobium Angustifolium and commonly found in all Epilobium species.

FIG. 4 is a listing of the most commonly known species of Epilobium known to the USDA.

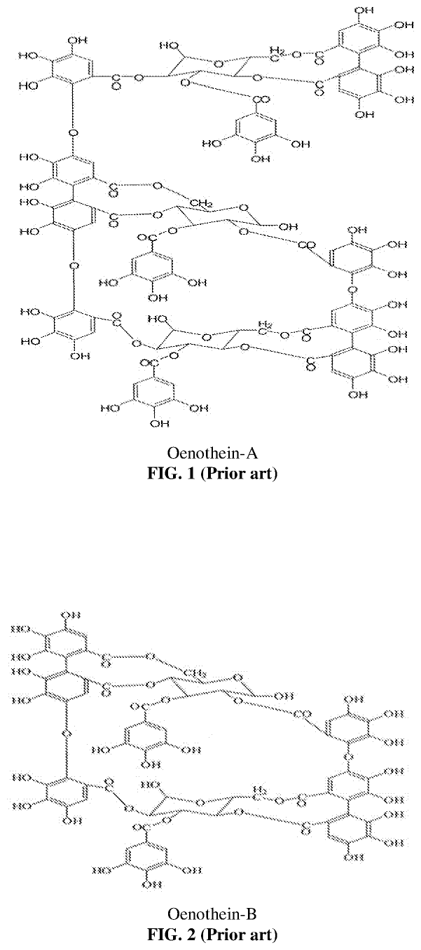

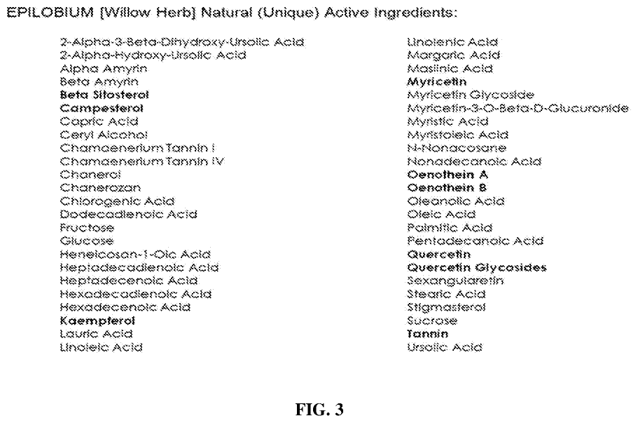

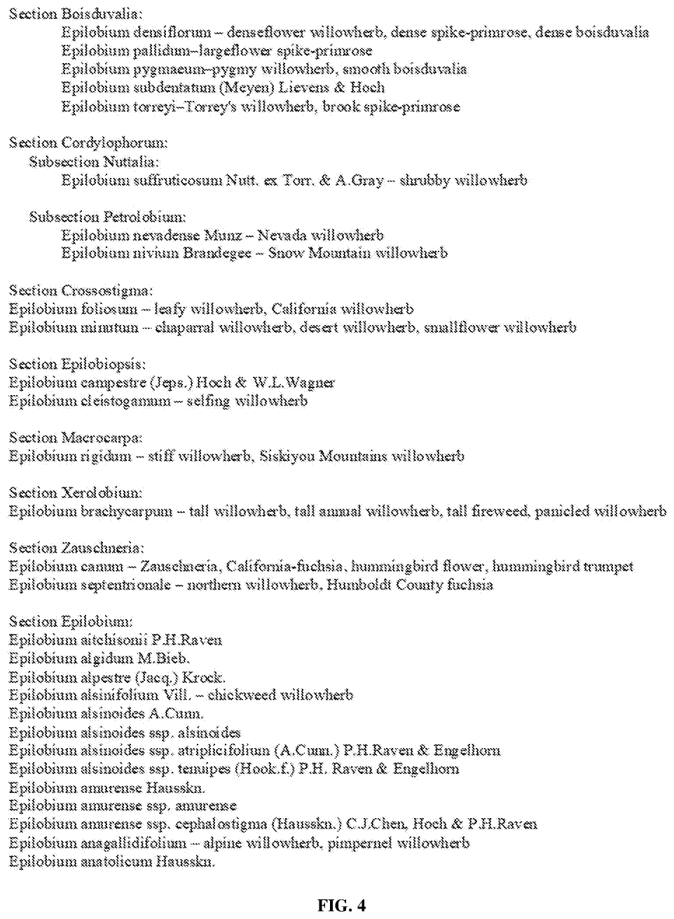

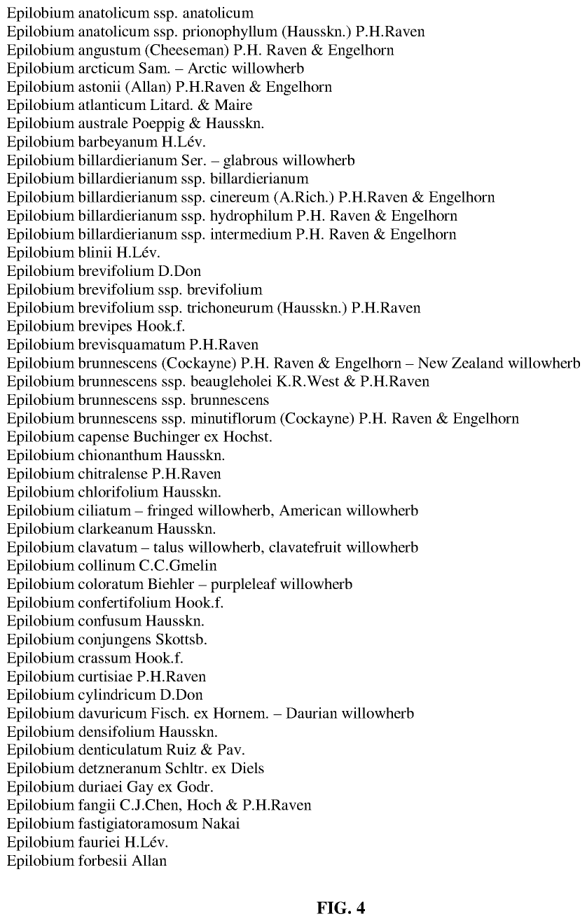

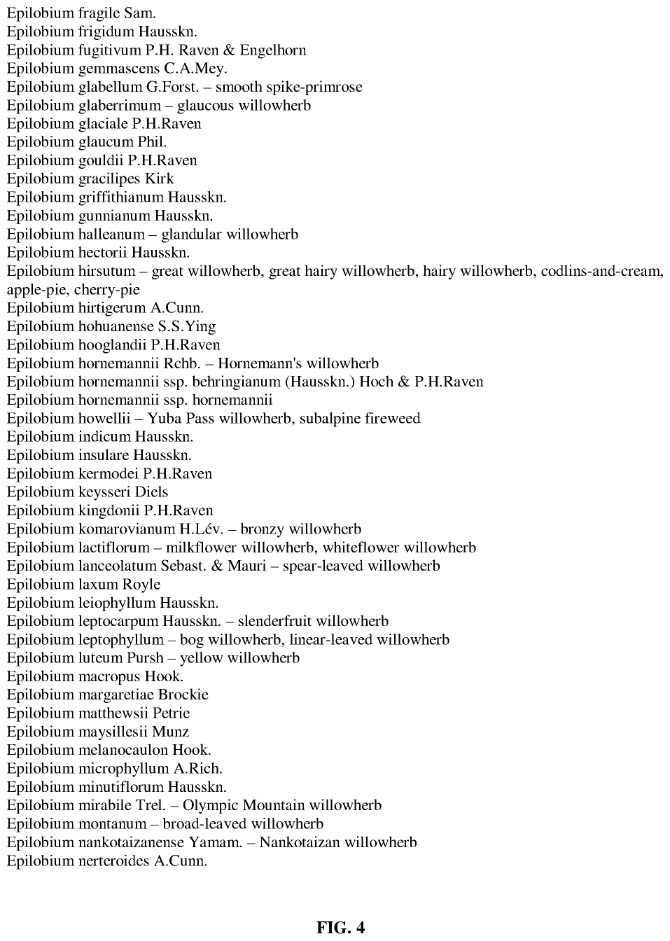

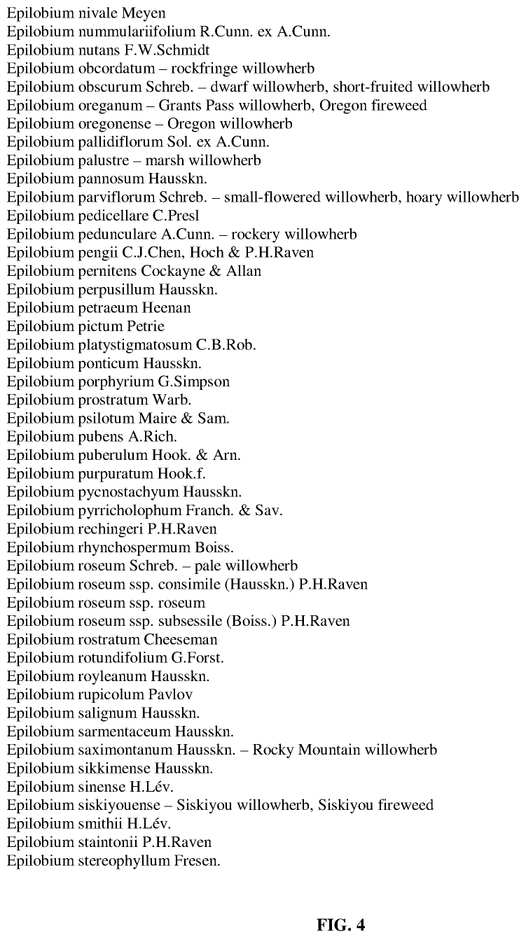

### DETAILED DESCRIPTION OF THE INVENTION

The present invention is based on the seminal discovery that the administration of oenothein A and B, associated with a cannabinoid is useful for treating a variety of disorders. It has been surprisingly discovered that oenothein's are potent anti-inflammatories, anti-irritant's and free radical scavengers which inhibit cyclooxygenase-1 and -2 (COX-1 and COX-2) catalyzed prostaglandin biosynthesis, accordingly, the invention provides formulations comprising an oenothein including orally administered for use as free radical scavengers or as prophylactic against damage caused by inflammation, hormone imbalance caused by the normal aging process or imbalance by environmental, biological, pathogenic or other influences. The oenothein for use in such formulations may for example be oenothein A or oenothein B. The oenothein maybe purified from natural sources, such as plant material, or it may be prepared synthetically.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of treating one or more symptoms of overactive bladder in a subject including administering to the subject a therapeutically effective amount of a composition including oenothein A or and B, and a cannabinoid, wherein the one or more symptoms of overactive bladder are selected from the group consisting of frequency of or urgency, nocturia, increase in urinary micturition frequency, urinary incontinence, hormone balance for perimenopausal, menopausal, postmenopausal women and inflammation, and wherein the cannabinoid is cannabidiol (CBD), tetrahydrocannabinol (THC), cannabigerol (CBG), or a combination thereof.

As used herein “treating” refers to the administration of a treatment which refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In once aspect, a therapeutically effective amount comprises a daily administration of about 350 mg to about 400 mg of oenothein A and B, and about 0.05 mg to about 50 mg of CBD, about 1 mg to about 15 mg of THC, or about 0.05 mg to about 500 mg of CBG. In another aspect, a therapeutically effective amount to treat chronic symptoms comprises about 1000 mg of oenothein A and B, and about 1,000 mg to about 2,000 mg of CBD, about 30 mg to about 100 mg of THC, or about 500 mg to about 1000 mg of CBG.

A “therapeutically effective amount” refers to an amount effective, at dosages and for periods of time necessary, to achieve the desired therapeutic result, such as reduction or reversal of inflammation or irritation. A therapeutically effective amount of an the composition of the present invention may vary according to factors such as the disease state, age, sex, weight of the individual, and the ability of the oenothein to elicit a desired response in the individual. Dosage regimens maybe adjusted to provide the optimum therapeutic response. A therapeutically effective amount is also one in which any toxic or detrimental effects of the oenotheins and CBD, CBG, or THC are outweighed by the therapeutically beneficial effects.

A “prophylactically effective amount” refers to an amount effective, at dosages and for periods of time necessary, to achieve the desired prophylactic result, such as preventing or inhibiting the rate of inflammation or irritation or the onset of inflammation or irritation, or quenching of free radicals. A prophylactically effective amount can be determined as described above for the therapeutically effective amount.

In particular embodiments, a preferred range for therapeutically or prophylactically effective amounts of an oenothein maybe 2.5% to 14% by weight. Dosage values may vary with the severity of the condition. It is to be further understood that for any particular subject, specific dosage regimens maybe adjusted over time according to the individual need and the judgment of the person administering or supervising the administration of the compositions, and that dosage ranges set forth herein are exemplary only and are not intended to limit the scope or practice of the methods of the invention.

In some aspects, the oenothein A or B is formulated in an oral dosage form. In various aspects, the composition is formulated as a concentrated oil extracts infused sublingual spray, a tincture, an edible, a gel capsule, a pH-resistant capsule, a topical salve, drops, or an aerosol, a capsule, liquid.

Pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In one aspect, the subject is a human. In some aspects, the human is a female.

The term “subject” as used herein refers to any individual or patient to which the subject methods are performed. Generally the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

In another embodiment, the present invention provides a composition formulated for oral or topical delivery consisting essentially of oenothein A, oenothein B, and a cannabinoid, wherein the cannabinoid is cannabidiol (CBD), tetrahydrocannabinol (THC), cannabigerol (CBG), or a combination thereof.

Oenothein B (OeB) has quite low bioaccessibility for oral consumption due to its susceptibility to decomposition both in vitro and in vivo. Herein, the design and synthesis of food-grade polyelectrolyte complex coacervate using case in phosphopeptides (CPPs) and chitosan (CS) to encapsulate OeB for enhanced protection through gastrointestinal (GI) tract.

Oenothein can be extracted from crude aqueous extracts of New World Epilobium parviflorum (Small Willow herb), using various extraction methods including using steam, water, oils, alcohols, glycerol, ultrasounds (ultrasonification), CO₂, or dry ice.

Glycerol is a simple polyol compound, it is a colorless, odorless, viscous liquid that is sweet-tasting and non-toxic. The glycerol backbone is found in all lipids known as triglycerides. Many plants can be used as a source of glycerol, typical plant sources include soybeans or palm.

Cannabis, also known as marijuana is a psychoactive drug from the Cannabis plant used for medical or recreational purposes. Medical cannabis, or medical marijuana, can refer to the use of cannabis and its cannabinoids to treat disease or improve symptoms. The classical cannabinoids are concentrated in a viscous resin produced in structures known as glandular trichomes. At least 113 different cannabinoids have been isolated from the Cannabis plant. The best studied cannabinoids include tetrahydrocannabinol (THC), cannabidiol (CBD) and cannabinol (CBN).

Tetrahydrocannabinol (THC) is the primary psychoactive component of the Cannabis plant, found as delta-9-tetrahydrocannabinol (Δ9-THC, THC) and delta-8-tetrahydrocannabinol (Δ8-THC); which produce the effects associated with cannabis by binding to the CB1 cannabinoid receptors in the brain. There as some scientific evidence supporting the the effectiveness of the cannabis extracts in treating certain symptoms of multiple sclerosis and pain, especially centrally mediated pain and painful spasms. The effects of THC on various symptoms observed in neurodegenerative diseases and disorders, such as Huntington disease, Parkinson's disease, Alzheimer's disease or Tourette syndrome have been assessed in trials.

Cannabidiol (CBD) is a phytocannabinoid discovered in 1940. It accounts for up to 40% of the cannabis plant cannabinoid's extract. As of 2019, clinical research on cannabidiol included preliminary studies of anxiety, cognition, movement disorders, and pain, but there is insufficient, high-quality evidence that it is effective for these conditions. Cannabidiol can be taken into the body in multiple ways, including by inhalation of cannabis smoke or vapor, as an aerosol spray into the cheek, and by mouth. It may be supplied as CBD oil containing only CBD as the active ingredient (no included tetrahydrocannabinol [THC] or terpenes), a full-plant CBD-dominant hemp extract oil, capsules, dried cannabis, or as a prescription liquid solution. CBD does not have the same psychoactivity as THC, and may change the effects of THC on the body if both are present.

Cannabigerol (CBG) is the non-acidic form of cannabigerolic acid, the parent molecule from which other cannabinoids are synthesized.

During growth of the plant, most of the cannabigerol is converted into other cannabinoids, primarily tetrahydrocannabinol (THC) or cannabidiol (CBD), leaving about 1% cannabigerol in the plant, therefore, CBG is considered a minor constituent of cannabis. Contrary to the major psychoactive cannabinoid THC, CBG is non-psychoactive but still contributes to the overall effects of Cannabis

In some aspects, the composition is formulated as a concentrated oil extracts infused sublingual spray, a tincture, an edible, a gel capsule, a pH-resistant capsule, a topical salve, drops, or an aerosol. In other aspects, the composition is formulated for oral delivery in an oral dosage form.

In certain aspects, the composition optionally includes an agent selected from the group consisting of a pharmaceutically acceptable excipient, lubricant, bind, glidant, filler, flavoring agent, masking agent, vitamin, mineral, a carrier and mixtures thereof.

As used herein “pharmaceutically acceptable carrier” or “excipient” includes any and all creams, gels, solvents, dispersion media, sublingual, buccal administration, coatings, antibacterial, anti-pathogen and antifungal agents, isotonic and absorption delaying agents, and the like that are physiologically compatible and do not significantly adversely affect the pharmaceutical properties (e.g. toxicity and effectiveness) of the oenothein, such as are conventionally used in the cosmetic and pharmaceutical arts. In one embodiment, the carrier is suitable for oral administration. Under some conditions, oenothein's may undergo hydrolysis in acid or base, so that pharmaceutically acceptable carriers or excipients may include pH buffers to maintain an acceptable pH for pharmaceutical activity.

In one aspect, the composition is in an oral dosage form in a single dosage. In other aspects, the composition is in an oral dosage form in a single dose enteric coated formulation.

In various aspects, the oral dosage form is a liquid oral formulation.

In some aspects, the oenothein A and B, and the CBD, CBG, THC, or the combination thereof are formulated for oral delivery in a capsular form and contained in a package with instructions for use.

In an additional embodiment, the invention provides a method of reducing inflammation, treating urinary incontinence, treating bacterial urinary incontinence, treating hormonal imbalance in perimenopausal, menopausal and postmenopausal women, treating female hair loss due to perimenopause, menopause, and/or postmenopause, treating the conversion of testosterone to estrogen (aromatase) in perimenopausal, menopausal, postmenopausal women, inhibiting cyclooxygenase-1 and -2 (COX-1 and COX-2) catalyzed prostaglandin biosynthesis, including administering to a subject in need thereof a therapeutically effective amount of a composition consisting essentially of oenothein A, oenothein B, and CDB, CBG, THC, or a combination thereof.

The terms “inhibiting” or “reducing” or “preventing” or “avoiding” or any variation of these terms, when used in the claims and/or the specification includes any measurable decrease or complete inhibition to achieve a desired result.

The term “effective” as that term is used in the specification and/or claims, means adequate to accomplish a desired, expected, or intended result.

As used herein “aromatase” refers to an enzyme that is responsible for a key step in the biosynthesis of estrogens. Because estrogens also promote certain deadly pathologies and other diseases, aromatase inhibitors are frequently used to treat those diseases. Dihydrotestosterone (DHT) is a biologically active metabolite (byproduct) of the hormone testosterone; it is formed primarily in the prostate gland, testes, hair follicles, and adrenal glands by the enzyme 5-alpha-reductase. 5-alpha-reductaseisanenzyme involved in steroid (androgen and estrogen) metabolism.

In yet another embodiment, the invention provides a method of treating methicillin-resistant Staphylococcus aureus (MRSA) infection in a subject including administering to the subject a therapeutically effective amount of a composition consisting essentially of oenothein A, oenothein B, and cannabigerol (CBG).

Methicillin-resistant Staphylococcus aureus (MRSA) refers to a group of Gram-positive bacteria that are genetically distinct from other strains of Staphylococcus aureus.

Treatment of MRSA infection is urgent and delays can be fatal. Standard care usually includes the administration of antibiotics effective against MRSA, which can be by IV, oral, or a combination of both. Concurrent treatment with vancomycin or other beta-lactam agents may have a synergistic effect. Various antibacterial chemical extracts from various species of the sweetgum tree (genus Liquidambar) have been investigated for their activity in inhibiting MRSA. Specifically, these are: cinnamic acid, cinnamyl cinnamate, ethyl cinnamate, benzyl cinnamate, styrene, vanillin, cinnamyl alcohol, 2-phenylpropyl alcohol, and 3-phenylpropyl cinnamate.

In some aspects, a therapeutically effective amount comprises a daily administration of about 350 mg to about 400 mg of oenothein A and B, and about 0.05 mg to about 500 mg of CBG.

In other aspects, a therapeutically effective amount to treat chronic symptoms comprises about 1000 mg of oenothein A and B, and about 500 mg to about 1000 mg of CBG.

In one aspect, the invention provides formulations or medicaments for treating a variety of symptoms, diseases or disorders, including inflammation, such as inflammation and irritation of the urinary tract, to inhibit cyclooxygenase-1 and -2 (COX-1 and COX-2) catalyzed prostaglandin biosynthesis, neuropathy, as an antioxidant for free radical scavenging as a hormone balancing agent, oenothein B has an effect on hair loss for both men and women, hormone balancing for perimenopausal, menopausal and postmenopausal women's hair loss. As an antibacterial a wide variety diseases are treatable with the compounds of the invention, including Escherichia coli is one of the most frequent causes of many common bacterial infections, including cholecystitis, bacteremia, cholangitis, urinary tract infection (UTI) and traveler's diarrhea and other clinical infections such as neonatal meningitis and pneumonia. As an anti-fungal oenothein B interferes with the cell morphology of the fungus Paracoccidioides brasiliensis, the most prevalent human systemic mycosis in Latin America. (Therapeutic Potential of Polyphenols from Epilobium angustifolium, (Fireweed) Igor A. Schepetkin, 2017).

Aromatase inhibition, the conversation of testosterone to estrogen or according to the American Cancer Society (ACS), aromatase inhibitors work differently from tamoxifen and raloxifene. Instead of blocking the estrogen receptors, they stop a key enzyme (called aromatase) from changing other hormones into estrogen; this lowers estrogen levels in the body, taking away the fuel that estrogen receptor-positive breast cancers need to grow. ACS, (Jul. 20, 2013).

In some aspects, the invention provides methods of medical, therapeutic or prophylactic dose of an oenothein is administered, such as by administration of a pharmacologically acceptable formulation. Such formulations of the invention may comprise oenothein A or oenothein B and a pharmacologically acceptable excipient or carrier, and may comprise a pharmaceutically acceptable salt of the oenothein. In some embodiments, such formulations may comprise a therapeutically or prophylactically effective amount sufficient to alter, and preferably inhibit, inflammation or hormone imbalance, or to quench free radicals.

In various aspects, the oenothein A and B are extracted or prepared from New World Epilobium parviflorum (small willow herb) and purified to a concentration of more than 10% by weight. In other aspects, the oenothein A and B are extracted or prepared from a plant source other than New World Epilobium parviflorum.

In some aspects, the oenothein A and B are extracted or prepared from New World Epilobium parviflorum (small willow herb) and purified to a concentration of more than 5% by weight. In other aspects, the oenothein A and B are extracted or prepared from a plant source other than New World Epilobium parviflorum.

Other aspects of the invention include methods of formulating oral medication for treatment of inflammation, irritants or free radical scavengers, comprising adding a known amount of an oenothein A and B, and CBD, CBG, or THC to such compositions (as to be of greater effectiveness we are motivated to determine the amount of oenothein being administered in herbal remedies per malady i.e., as a DHT Blocker or Aromatase Inhibitors), such as in Epilobium parviflorum extracts. In light of past and the present inventions, plant extracts for use in such formulations may now be assayed (Sigma Aldrich), to determine the concentration of oenothein B in the extracts so that dosages maybe formulated with a higher level of reliably. For example, New World Epilobium parviflorum maybe assayed for oenothein B content. Packaging for formulations of the invention may include text that indicates that the formulations are useful for prophylactic inflammation due to strenuous activity, exposure to free radicals or hormone imbalance, such text may optionally disclose that the formulations contain oenothein B.

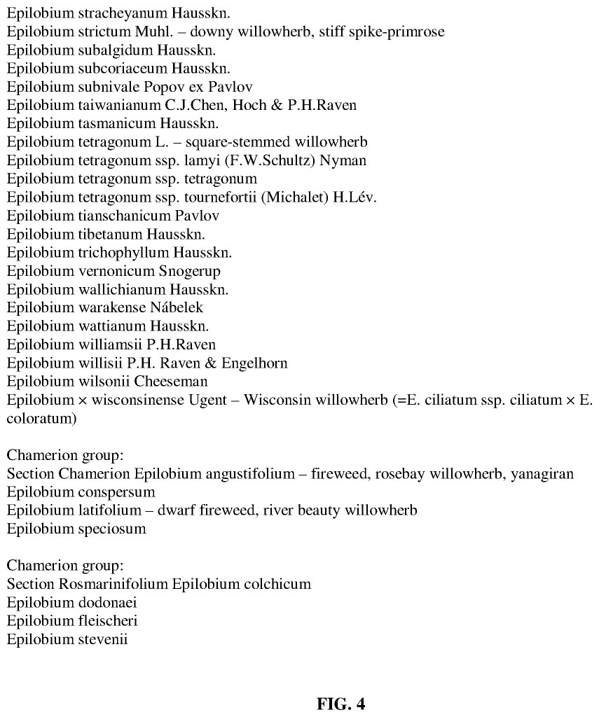

In another aspect of the invention some of the known species of Epilobium (see FIG. 4) contain oenothein and may be orally administered as an anti-inflammatory, free radical scavengers or for treating hormone imbalance. For example, Epilobium E. rosmarinifolium and Epilobium E. angustifolium may be used for making such extracts where they contain oenothein A or oenothein B or mixtures thereof. It is also contemplated that compositions of the present invention oenothein A and or B can be included into functional food or beverage products (e.g., alcoholic beverages, fortified water, energy drinks, nutritional drinks, vitamins, supplements, functional foods and other ingestible variations) and pharmaceutical products (e.g. pills, injectable solutions). “Supplements” can include vitamins, minerals, herbs or the botanicals, amino acids, enzymes and metabolites. Such supplements can be administered orally.

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The ingredients/components can also exist as un-dissolved colloidal suspensions.

In accordance with another aspect of the invention, therapeutic compositions of the present invention, comprising an oenothein, maybe provided in container labels that provide instructions for use of the formulation to: treat inflammation, hormone imbalance, scavenge free radicals, urinary incontinence. The labels may also disclose that the compositions comprise an oenothein.

It has previously been disclosed, by the present inventor, that crude aqueous extracts of Epilobium parviflorum have anti-inflammatory, hormone balancing and free radical scavenging properties when taken orally. In accordance with the present invention the concentration of oenothein B in such extracts exceed <5% by weight. Accordingly, with the unexpected discovery that oenothein B is an active ingredient in such extracts, the present invention provides for novel formulations of Epilobium parviflorum or other Epilobium species extracts having oenothein B concentrations in excess of <5%. In alternative aspects, the present invention provides formulations for treating inflammation, hormone imbalance or free radical scavenging that are prepared from purified oenothein B obtained from Epilobium parviflorum or other Epilobium species, where the oenothein B is purified to a concentration of greater than <5% prior to formulation. In alternative embodiments, purified oenothein B from Epilobium parviflorum or other Epilobium species may be utilized in the present invention in concentrations ranging from 1%<up to concentrations of approximately 100%.

A variety of methods may be used to purify oenothein from natural sources for use in the various aspects of the present invention. For example, U.S. Pat. No. 5,525,594 (incorporated herein by reference) discloses methods of preparing oenothein B from plants. Similarly, Ducrey et al., 1996, supra disclose oenothein A and oenothein B purification and characterization methods (incorporated herein by reference). Alternative purification methods may be used in accordance with the present invention, provided that they may be used to produce a pharmaceutically acceptable preparation of purified oenothein B suitable for use in the various aspects of the present invention.

Presented below are examples discussing the administration of oenothein A or B contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Efficacy of Oenothein B to Treat Inflammation and Irritation

A purified aqueous extract was obtained from New World Epilobium angustifolium (Canadian Willow herb) and subjected to a variety of analytical techniques, which unambiguously identify the purified compound as oenothein B. This analysis included mass spectroscopy and nuclear magnetic resonance spectrometry. While oenothein B was found by these methods to be present in solvent-free aqueous extracts of Canadian Willow herb at levels up to 9.6%, myricetin glucuronide—the substance identified as the active ingredient of the anti-inflammatory action of Old World Epilobium angustifolium (U.S. Pat. No. 6,528,490 B2, see the Background section herein)—was not found in extracts of Canadian Willow herb.

### Example 2

### Efficacy of Oenothein B to Treat Inflammation and Irritation

This example shows the effectiveness of the purified oenothein B characterized in Example 1 in treating inflammation and irritation.

In the hen's-egg chorioallantoic membrane (CAM) model, in which the CAM is treated with 15% lactic acid as a standard irritant, the development of manifestations of membrane irritation were tracked and scored according to published methods for assessing irritancy. A sample of the oenothein B extract from Canadian Willow herb described in Example 1 was tested at a 1% (weight to volume) dilution for activity in the CAM model. The results showed a decrease in irritation by up to 70% when the oenothein B was applied before the irritant (simulating a prophylactic use) while an 80% decrease was seen with the whole aqueous extract itself.

Further testing was performed with whole aqueous extract of Canadian Willow herb and a whole extract sample spiked with 10% additional oenothein B. The results showed an increase in the reduction of irritation by the spiked sample of 19% over that of the whole extract by itself.

In human skin patch tests, an oenothein B extract from Canadian Willow herb formulated in a lotion ameliorated the irritant effects of 15% lactic acid, as measured after 0.5, 1, 4 and 24 hours, compared to controls with no treatment, treatment with water and treatment with the lotion alone (containing no extract).

### Example 3

### Free Radical Scavenging Activity of Oenothein B

This example shows the free radical scavenging activity of oenothein B. A sample of the oenothein B extract from Canadian Willow herb described in Example 1 was assessed using the xanthine oxidase/acetaldehyde spectro photometric method, in which superoxide radical ions regenerated in vitro using acetaldehyde and xanthine oxidase, in the presence of test compounds. The scavenging of free super oxide radicals is quantitated spectrometrically and expressed in terms of percentage of radicals scavenged. Assays were performed with 1% (w/v) and 0.1% (w/v) dilutions of the dried extract, which yielded up to 100% scavenging activity and up to 99% scavenging activity respectively.

### Example 4

### Quantifying Oenothein B

This example shows Epilobium parviflorum using a method of quantifying oenothein B using Gallic Acid (Hydrolyzed) and Gallic Acid (Free). After using the Gallic Acid method on Epilobium parviflorum the following values were recorded 0.0074, 0.0087 and 0.0096. Ellagitannins are usually separated and characterized by HPLC, HPLC-MS, MALDI-TOF-MS, and HPTLC [17-20]. There is currently a method for using oenothein B through a marker made available by Sigma Aldrich.

### Example 5

### Efficacy of Epilobioum parviflorum Extract on Prostate Inflammation

This example shows the effectiveness of 200 mg sample of Epilobium parviflorum extracted with sugar cane alcohol and purified water. A study was done with a difficult elderly population in generally poor health, with the participants taking multiple prescription medications. The average age of the participants was (82 years of age). In spite of this, the results were quite remarkable in that 55.6% (5 out of 9) improved significantly, with an average improvement of 48.11%, 11.1% (1 out of 9) showed no change, 33.3% (3 out of 9) worsened with an average worsening of 19.7%. Given this difficult sample it is likely that this population would have worsened anyway with or without intervention. The focus of the study was prostate inflammation causing frequent trips to the bathroom. All the patients were under the care of Dr. Daniel L. Johnson, M. D., Desert Longevity Institute, Palm Desert, Calif.

### Example 6

### Efficacy of Epilobioum parviflorum Extract in Combination with Cannabinoids

Novel products comprising Oenothein A and B extracted from Epilobium in combination with Cannabidiol (CBD), cannibogerol (CBG), or tetrahydrocannabinol (THC) will be developed, and their efficacy to treat symptoms of overactive bladder such as frequency or urgency, nocturia, increase in urinary micturition frequency, urinary incontinence, hormone balance for perimenopausal, menopausal, postmenopausal women and inflammation will be evaluated.

The compositions will also be evaluated for their efficacy to reduce inflammation, treat urinary incontinence, treat bacterial urinary incontinence, treat hormonal imbalance in perimenopausal, menopausal and postmenopausal women, treat female hair loss due to perimenopause, menopause, and/or postmenopause, treat the conversion of testosterone to estrogen (aromatase) in perimenopausal, menopausal, postmenopausal women, and to inhibit cyclooxygenase-1 and -2 (COX-1 and COX-2) catalyzed prostaglandin biosynthesis.

Oenothein A and B will be extracted from Epilobium using one of the various extraction methods available for their efficient extraction, such as using steam, water, oils, alcohols, glycerol, ultrasonification), CO₂, or dry ice.

The products will be prepared in various delivery forms, such that a user can chose between the delivery form that suits better its habits, and that will ensure an optimal observance of the treatment. For example, the products will be delivered as concentrated oil extracts infused sublingual sprays, tinctures, edibles, gel capsules, pH-resistant capsules, topical salves, drops, or aerosols.

Depending on the disease or disorder, and on the stage, various dosages will be recommended. For chronic affections, a daily dosage of 1,000 mg of Epilobium, combined with 1,000 mg-2,000 mg CBD, 500-1000 mg CBG, or 30-100 mg THC will be recommended. For non-chronic affection, of for prophylactic purposes, a daily dosage of 350-400 mg of Epilobium, combined with 0.05 to 50 mg CBD, 0.05-500 mg CBG, or 1 mg-15 mg THC will be recommended. These dosages would be a combination of Oneothein A and B and CBD, CBG or THC as a combined product.

### Example 7

### Efficacy of Epilobioum parviflorum Extract in Combination with Cannibogerol to Treat MRSA Infection

A composition comprising Oenothein A and B extracted from Epilobium in combination with cannibogerol (CBG), will be developed; and its efficacy to treat methicillin-resistant Staphylococcus aureus (MRSA) infection will be evaluated.

The product will be prepared in various delivery forms, such that a user can chose between the delivery form that suits better its habits, and that will ensure an optimal observance of the treatment. For example, the product will be delivered as concentrated oil extracts infused sublingual sprays, tinctures, edibles, gel capsules, pH-resistant capsules, topical salves, drops, or aerosols.

Depending on the stage, various dosages will be recommended. For chronic infection, a daily dosage of 1,000 mg of Epilobium, combined with 500-1000 mg CBG will be recommended. For non-chronic infection, of for prophylactic purposes, a daily dosage of 350-400 mg of Epilobium, combined with 0.05-500 mg CBG, will be recommended. These dosages would be a combination of Oneothein A and B and CBG as a combined product.

Those of skill in the art should, in light of the present disclosure, appreciate that many changes can be made in the specific embodiments which are disclosed and still obtain alike or similar result without departing from the spirit and scope of the invention. The present invention is not to be limited in scope by the specific embodiments described herein, which are intended as illustrations of individual aspects of the invention, and functionally equivalent methods and components are within the scope of the invention. Indeed, various modifications of the invention, in addition to those shown and described herein, will become apparent to those skilled in the art from the foregoing description. Accordingly, the invention is limited only by the following claims.

### REFERENCES

1. Aoki, K. et al. (1995), Biochem Biophys Res Commun, A Macro circular Ellagitannin, Oenothein B., Suppresses Mouse Mammary Tumor Gene Expression via Inhibition of Poly(ADP-ribose) Glycohydrolase, 210(2): 329-37.
2. Dijkstra et al. (1996). “Repertorium 96/97” SDUService-centrumUitgeveverijen.
3. Ducrey et al. “Inhibition of 5-alpha-reductaseandaromatasebythe ellagitannins oenothein A and oenothein B from Epilobium species. “Planta Med. (1977), 63(2), 111-114.
4. Ducrey et al. (1997). “Inhibition of 5-alpha-reductase and aromatase by the ellagitannins oenothein A and oenothein B from Epilobium species “Planta Medica, vol. 63(2): 111-
5. Hendry et al. “Iron-induced oxygen radical metabolism in waterlogged plants”. HCAPLUS103:211253, New Phytol. (1985), 101 (1), 199-206.*
6. Hiermann (1987). “The investigation of active compounds from Epilobium species. 4th Communication. The anti-inflammatory effect of different Epilobium species and their influence on prostaglandin “Scientia Pharamaceutica, vol. 55(2): 111-116.
7. Hiermann, et al. (1991), PlantaMedica, 57:357.
8. Juan et al. “Anti-inflammatory effects of a substance extracted from Epilobium angustifolium”. Medline: 88180554 Agents and Actions, (1988 February) 23 (1-2), 106-
9. Kotaro (1997). “Anti allergy action of Rubus suavissimus” ChemicalAbstracts, vol. 127(9):52-59.
10. Lesuisse, D. et al. (1996), J Nat Prod, Determination of Oenothein Bas the Active 5-Alpha-Reductase-Inhibiting Principle of the Folk Medicine Epilobium parviflorum,
11. Luepke, N. P. & Kemper, F. H., (1986), The HETCAM Test: An Alternative to the Draize Eye Test, Food and Chemical Toxicology,
12. Miyamoto, K., et al. (1993), Jpn J Cancer Res, Antitumor Activity of Oenothein B., a Unique Macroyclic Ellagitannin, 84(1):99-103.
13. Okuda, T., Yoshida, T. & Hatano, T. (1989), Ellagitannins as Active Constituents of Medicinal Plants, Planta Med, 55(2):117-22.
14. Spielmann et al., (1997), CAM-based Assays, Food and Chemical Toxicology, 35:39-66.
15. Polyphenols from Bulgarian medicinal plants with anti-infectious activity. Ivancheva S; Manolova N; Serkedjieva J; Dimov V; Ivanovska N. Basic Life Sci, 590:717-28
16. A Kiss, J Kowalski, M F Melzig. Induction of neutral endopeptidase activity in PC-3 cells
17. By an aqueous extract of Epilobium angustifolium L. and oenothein B. Affiliation: Department of Pharmacognosy, Medical University of Warsaw, and Warsaw, Poland. Kiss ankaat wp.pl) Journal Phytomedicine: International Journal of Phototherapy and Phytopharmacology (Phytomedicine) Vol. 13 Issue 4 Pg. 284-9 (March 2006)
18. Studies on antibacterial, anti-inflammatory and antioxidant activity of herbal remedies used in the treatment of benign prostatic hyperplasia and prostatitis. Steenkamp, Vanessa; Gouws, M. C.; Gulumian, M.; Elgorashi, E. E.; VanStaden, J. (2006)
19. Improved and Validated HPTLC Method for Quantification of Oenothein B and its Use for Analysis of Epilobium angustifolium L. Alexander N. Shikov*, Olga N. Pozharitskaya, Svetlana A. Ivanova, Valery G. Makarov, Vladimir P. Tikhonov, and Bertalan Galambosi
20. Immunomodulatory Activity of Oenothein B Isolated from Epilobium angustifolium Igor A. Schepetkin*, Liliya N. Kirpotina*, Larissa Jakiw*, Andreil. Khlebnikov.dagger., Christie L. Blaskovich*, Mark A. Jutila*, and Mark T. Quinn*, 2*Departments of Veterinary Molecular Biology, Montana State University, Bozeman, Mont. 59717 .dagger.Department of Chemistry, Altai State Technical University, Barnaul 656038, Russia
21. The vascular protective effects of estrogen. Farhat M Y.sup.1, Lavigne M C, Ramwell P W
22. Phytochemistry, pharmacology and traditional uses of different Epilobium species. (Onagraceae) 2014, Kiss A
23. Assessment report on Epilobium angustifolium L. and/or Epilobium parviflum Schreb., herba (Final) European Medicines Agency 2016
24. Topical Melatonin for treatment of Androgenic Alopecia
25. Androgenetic Alopecia-A Complex Disorder—with a Multilateral Treatment Strategy, 2017
26. Rational design of food-grade polyelectrolyte complex coacervate for encapsulation and oral enhanced oral delivery of oenothein B. Lan Y 2017
27. Prevalence and Trends of Urinary Incontinence in Adults in the United States, 2006-2010
28. Therapeutic Potential of Polyphenols from Epilobium angustifolium (Fireweed) Igor A. Schepetkin, Andrew G. Ramstead, Liliya N. Kirpotina, Jovanka M. Voyich, Mark A. Jutila, and Mark T. Quinn
29. Antibacterial activity of silver nanoparticles: A surface science insight, 2016, Benjamin Le Ouay Francesco Stellacci
30. Wikipedia (glycerol)

What is claimed is:

1. A method of treating one or more symptoms of overactive bladder in a subject comprising administering to the subject a therapeutically effective amount of a composition comprising oenothein A and B, and a cannabinoid,
wherein the one or more symptoms of overactive bladder are selected from the group consisting of frequency or urgency, nocturia, increase in urinary micturition frequency, urinary incontinence, hormone balance for perimenopausal, menopausal, postmenopausal women and inflammation, and
wherein the cannabinoid is cannabidiol (CBD), tetrahydrocannabinol (THC), cannabigerol (CBG), or a combination thereof.

2. The method of claim 1, wherein a therapeutically effective amount comprises a daily administration of about 350 mg to about 400 mg of oenothein A and B, and about 0.05 mg to about 50 mg of CBD, about 1 mg to about 15 mg of THC, or about 0.05 mg to about 500 mg of CBG.

3. The method of claim 1, wherein a therapeutically effective amount to treat chronic symptoms comprises about 1000 mg of oenothein A and B, and about 1,000 mg to about 2,000 mg of CBD, about 30 mg to about 100 mg of THC, or about 500 mg to about 1000 mg of CBG.

4. The method of claim 1, wherein the composition is formulated as a concentrated oil extracts infused sublingual spray, a tincture, an edible, a gel capsule, a pH-resistant capsule, a topical salve, drops, or an aerosol.

5. The method of claim 1, wherein the subject is a human.

6. The method of claim 4, wherein the human is a female.

7. A composition formulated for oral or topical delivery consisting essentially of oenothein A, oenothein B, and a cannabinoid,
wherein the cannabinoid is cannabidiol (CBD), tetrahydrocannabinol (THC), cannabigerol (CBG), or a combination thereof.

8. The composition of claim 7, wherein the composition is formulated as a concentrated oil extracts infused sublingual spray, a tincture, an edible, a gel capsule, a pH-resistant capsule, a topical salve, drops, or an aerosol.

9. The composition of claim 7, wherein the composition is formulated for oral delivery in an oral dosage form.

10. The composition of claim 7, wherein the composition optionally includes an agent selected from the group consisting of a pharmaceutically acceptable excipient, lubricant, bind, glidant, filler, flavoring agent, masking agent, vitamin, mineral, a carrier and mixtures thereof.

11. The composition of claim 9, wherein the composition is in an oral dosage form in a single dosage.

12. The composition of claim 9, wherein the composition is in an oral dosage form in a single dose enteric coated formulation.

13. The composition of claim 7, wherein the oenothein A and B, and the CBD, THC, CBG or the combination thereof are formulated for oral delivery in a capsular form and contained in a package with instructions for use.

14. A method of reducing inflammation, treating urinary incontinence, treating bacterial urinary incontinence, treating hormonal imbalance in perimenopausal, menopausal and postmenopausal women, treating female hair loss due to perimenopause, menopause, and/or postmenopause, treating the conversion of testosterone to estrogen (aromatase) in perimenopausal, menopausal, postmenopausal women, inhibiting cyclooxygenase-1 and -2 (COX-1 and COX-2) catalyzed prostaglandin biosynthesis, comprising administering to a subject in need thereof a therapeutically effective amount of the composition of claim 7.

15. A method of treating methicillin-resistant Staphylococcus aureus (MRSA) infection in a subject including administering to the subject a therapeutically effective amount of a composition consisting essentially of oenothein A, oenothein B, and cannabigerol (CBG).

16. The method of claim 15, wherein a therapeutically effective amount comprises a daily administration of about 350 mg to about 400 mg of oenothein A and B, and about 0.05 mg to about 500 mg of CBG.

17. The method of claim 15, wherein a therapeutically effective amount to treat chronic symptoms comprises about 1000 mg of oenothein A and B, and about 500 mg to about 1000 mg of CBG.

18. The method of claim 1, 14, or 15 wherein the oenothein A and B are extracted or prepared from New World Epilobium parviflorum (small willow herb) and purified to a concentration of more than 10% by weight.

19. The method of claim 1, 14 or 15, wherein the oenothein A and B are extracted or prepared from a plant source other than New World Epilobium parviflorum.

20. The composition of claim 7, wherein the oenothein is extracted or prepared from New World Epilobium parviflorum (small willow herb) and purified to a concentration of more than 5% by weight.

21. The composition of claim 7, wherein the oenothein is extracted or prepared from a plant source other than New World Epilobium parviflorum.
